(19) United States
(12) Patent Application Publication
(10) Pub. No.: US 2010/0318419 A1
(43) Pub. Date: Dec. 16, 2010

(54) PAY PER AUDIENCE FOR DIGITAL SIGNAGE ADVERTISING

(76) Inventor: Riccardo Vieri, Montespertoli (IT)

Correspondence Address: LOUIS J. HOFFMAN, P.C., 11811 North Tatum Boulevard, Suite 2100, Phoenix, AZ, 85028, US

(21) Appl. No.: 12/814,381

(22) Filed: Jun. 11, 2010

(30) Foreign Application Priority Data
Jun. 12, 2009 (IT) PO20090006

(51) Int. Cl. G06Q 30/00; G06Q 10/00; A61L 9/04

(52) U.S. Cl. 705/14.45; 239/34

(57) ABSTRACT

A “pay-per-audience” system allows advertisers to pay for advertising based on a number and a specified target of hits deemed as reliable, the system being manageable remotely.

### CROSS-REFERENCE TO RELATED APPLICATIONS

This application claims the benefit of Italian Patent Application No. PO20090006, filed on Jun. 12, 2009, which is hereby incorporated by reference in its entirety.

### BACKGROUND OF THE INVENTION

1. Field of the Invention

The present disclosure relates to the field of digital advertising and, particularly, it relates to the management of advertising campaigns wherein the costs of the advertising messages are charged based on a target, a number of hits, and logic remotely defined by an advertiser.

2. Description of Related Art

There are several methods for delivering advertising messages to terminals adapted for the visualization of images. Time spent at home is increasingly low, and so is the probability of reaching people through the most common communication media. In contrast, advertising on smart phones and mobile phones is experiencing increased interest.

Recently, another increasingly popular strategy is the installation of screens at airports, train stations, and high-traffic commercial installations to broadcast information, news, traffic reports, and meteorological forecasts. In this way, it is possible both to provide a useful service and, concurrently, to present advertising messages. Unfortunately, these indoor digital signage systems are unattractive for small local advertisers due to the absence of useful data concerning both the number of hits per day and the potential targets. Furthermore, the advertising messages are sold at a fixed price according to a cost-per-view model.

With the Internet revolutionizing the interpretation of advertising costs following the introduction of pay-per-click strategies, the offering of advertising solutions that cannot be measured and at a fixed price is not attractive and not forward-looking. For advertising through television, press, and radio, there are sufficiently reliable statistical data that can be used to determine the number of users per day or per average minute, as well as to trace a profile of the users. For digital advertising delivered to remote terminals, it is possible to provide transmission schedules for the advertising message and detailed reports on the locations to which the advertising message is delivered, and to trace a profile of the users.

There is a need for a system that allows advertisers to take advantage of new mechanisms to remotely create an advertising campaign and determine a number of a reliable cost per advertisement according to a specified target. There is a need for an alternative to the Internet that can identify a target searched for by an advertiser without collecting any personal information from the potential “captured” users, which personal information is, however, often unreliable in spite of the filling of forms or profiles. There is a need for an attractive form of advertising that can hit and identify a “moving” target while preserving the privacy thereof (in contrast with advertising on the Internet), particularly because the time spent at home is increasingly low.

Statistics for television advertising are merely indicative. For example, some families are monitored to obtain rating data associated with television broadcasters. It is possible to know which television programs are presented, when a television channel is changed and other details, but it is impossible to know if tele-viewers are actually present. Furthermore, this raises a privacy issue because each tele-viewer is provided with a code. Internet advertising allows both counting the number of visitors to a Web site and identification of the visitors clicking an advertisement, but it cannot determine what a visitor has effectively viewed on a given area of a Web page. Other media such as press and radio cannot generate feedback data with a similar degree of accuracy.

These forms of advertising cannot identify a well-defined age target. Even on the Internet, anyone can search for a term, for example the word ‘car’, but the advertising engine will display corresponding advertisements to all the searching users between 16 and 80 of age without discriminating among them, so that the yield of the advertisement will be lower than that expected. The present invention overcomes these difficulties by providing a solution having important effects for the advertising market.

In the past, sounds have been studied having particular frequencies which can be heard only by, for example, insects, or animals, for a variety of uses. It has been observed that certain sounds at particular frequencies can only be perceived by certain species. For example, electronic ultrasound devices are sometimes employed to repel insects and rodents. Recently, further studies have identified sounds that can be heard by human beings and that can be optimally perceived by certain age groups. For example, some sounds have been observed to be audible only to people under 18 years old, and others only to people over 25 years old.

Aspects of the invention address another important technical issue: the viewing angle of a conventional webcam is limited. To obtain appreciable results for a viewing angle of interest, video cameras provided with a detection system have been usually mounted on the ceiling.

In certain facilities, buzzing or other background noises can be annoying, or the surrounding silence can allow people to hear conversations meant to be confidential. However, privacy is critical in certain environments (a bank or medical facility, for example). Sound masking can be explained using an analogy of light and vision. A lamp is continuously switched on and off in a dark room would be constantly perceived and also unpleasant. Instead, if other lights are switched on, the lamp switching on and off is not perceived as readily. Sound masking works in an analogous manner to divert the attention using another sound which is not unpleasant and intrusive.

### SUMMARY OF THE INVENTION

The object of the present invention is to provide a “pay-per-audience” system that allows advertisers to pay for advertising based on a number and a specified target of hits deemed as reliable, the system being manageable remotely, having a higher degree of reliability than the current systems, and being able to meet the approval of advertisers on the market.

In a particular embodiment an advertiser is able to set a maximum cost per hit in order to ensure that his/her advertising message will be delivered with priority over possible competitors who have selected a lower cost per hit.

Furthermore, an advertiser can also set a cost per hit based on age characteristics of the visitor. For example, a company promoting videogames can establish that, if an advertisement is displayed to teen-agers under 16 years of age, then there would be a given cost per hit, but if it is displayed to older users, then such a cost per hit would be decreased.

In a particular embodiment the system discloses generation of statistical reports that can be produced in real time and viewed remotely, said reports concerning the effective charges applied to advertisers based on a target and a number of hits obtained from the remote terminals.

In a particular embodiment the system also discloses the ability to use Bluetooth® technology as a detection tool to count the number of attendees and to compare it with that detected by an optional video camera. Therefore, the system can detect all the owners of mobile phones within a certain range from the receiver apparatus of the terminal. Acquired data are saved and then compared with those from an image detection system. Because Bluetooth® devices can be identified by their codes, it is possible to ignore the code of a device either frequently present in a facility during one day or staying too long in the same facility, so as to improve the overall statistics.

The analysis from the Bluetooth® system can be combined with the analysis from the video camera to extrapolate definitive data in terms of the number of effective hits, at which time the cost set by the advertiser is charged.

The Bluetooth® connection can also provide for the ability to perform actions of proximity marketing. For example, the terminal can be configured to receive and verify the number of mobile phones present within a certain range, as well as to transmit preset advertising information, by transferring such data via Bluetooth® to all the individuals who are in the proximity of the terminal and recognized as eligible to receive a certain message. In addition to presenting the advertisements on those terminals, an advertiser can set a cost per hit for a message to be sent to the mobile phones of individuals identified as eligible by the system (for example, all the individuals having watched the screen of the terminal or only the individuals who are adult in age, etc.). In this specific case, the invention can be characterized not only by the analysis and determination of the number of hits, but also by a potential interaction therewith that occurs through other means external to the terminal.

In a particular embodiment, the system discloses a feature that aims to stimulate the interest of a specified age target in visualizing advertisements, based on the use of certain sounds having frequencies which can be heard preferentially by such a specified age target.

An advertisement can be provided with a sound for the first seconds of appearing. For example, a company promoting videogames can add audio to its ads is audible preferentially to people below 24 years of age. When the ads are shown on remote terminals, the sound would be heard only by desired targets, who would then turn and watch the advertisement on the terminal. The sound would attract the attention of the target age group without disturbing people of differing ages.

The detection and analysis of the individuals who are present when a message is presented are carried out by one or more devices connected to the remote terminal. Attendees are identified by a software program containing specialized algorithms; the software can also include the ability to recognize attendees by age characteristics and gender. If an advertiser selects such a criterion, the advertising message can be delivered according to the type of attendees. For example, if there are only male attendees near the terminal at a given time, the system can present a targeted advertisement as planned by an advertiser, based on a schedule which is changed according to who is in the proximity of the terminal. However, the same type of analysis allows the advertiser to effectively pay a cost that is established according to the criteria specified by the advertiser, such as for example age and/or gender. Similarly, it is possible to count the attendees who visualize a certain form of advertising. Furthermore, by analyzing the facility, it is also possible to verify whether the information provided by the owner of the facility, such as for example type of business, approximate number of hits, etc., are accurate or unreliable.

In a particular embodiment, one apparatus can both deliver advertising messages and carry out the analysis of the hits, thereby eliminating use of external devices that may not function because, for example, they run operating systems that are different from the operating system of the apparatus. An apparatus having this dual function simplifies mounting and installation and reduces costs, making feasible the use of a system for local and national advertising campaigns previously underutilized due to the difficulty of guaranteeing a certain number of reliable hits for the advertiser.

In a particular embodiment, the apparatus includes the use of a video camera integrated in the screen so that each video camera used can have a viewing angle of up to 170°, without ceiling mounting or wires, thereby reducing complications for the hosting facility.

In a particular embodiment, the video camera can be oriented and/or adjusted by means of a remote control and/or from a remote location, thereby simplifying the settings for visualization of the images, because orientation can be facilitated and made automatically. If the apparatus is located overhead and/or at a relatively inaccessible position, to control the image-capturing angle, the apparatus can be provided with a wireless monitor, optionally reduced in size, which allows the owner of the shop to visualize and orient the angle according to the most suitable parameters.

In a particular embodiment, the system can change the advertisements presented depending from the available target. For example, an advertiser can plan an advertising campaign so that one advertisement would be presented only when men are in proximity to the terminal, and another, different advertisement would be presented only when women are in proximity to the terminal. This feature can include several target options and allow the system to display “intelligent” advertisements only to the desired target.

In a particular embodiment an apparatus includes an audio-generating module to output a specified audio clip when a particular target is present. For example, if an advertiser wants to promote a concert of a band worshipped by female teenagers, the advertiser can give instructions for playing an advertising audio-clip promoting the concert when at least 5 girls under 18 years in age are in the proximity of the terminal.

In a particular embodiment, a method provides benefits to the managers of facilities hosting the apparatus. Typically, the owner of a hosting facility will receive a news service in exchange for the visualization of advertisements. The hosting facility can also receive other important benefits from the use of this specific apparatus. For example, the apparatus can include a sound generation device that generates sounds at particularly frequencies to create a “sound masking” effect. The apparatus can include a device that outputs sounds either through loudspeakers positioned at suitable locations or directly through the loudspeakers of the apparatus, such sounds being background sounds that are not unpleasant and that mask or prevent the recognition of spoken conversations by third parties. According to the present system, such a sound is generated continuously, on demand by the owner of the facility, or simultaneously with the visualization of an advertisement. This embodiment optionally includes a sound that can be heard preferentially by individuals belonging to specified age groups, a further advancement in the art to promote the spreading of this type of advertising, which is useful for the advertisers to obtain optimal results and which provides tangible benefits both to the owners of hosting facilities and to attendees.

In a particular embodiment an apparatus includes the use of olfactory marketing and/or environmental cleaning solutions. The apparatus of the present patent can include an ionic air purifier of any of various kinds, which can be integrated in or connectable to the apparatus, e.g., through a USB port. The air ionizers and purifiers can purify the air of facilities over some tens of square meters in the proximity of the same apparatus. In addition, the apparatus can also refresh it with one or more suitable fragrances. The hosting facility can benefit from air that is cleaner, healthier, and/or more pleasant, and advertising communication can be further boosted because certain fragrances have been shown both to increase the attention and to sensitize those interested in it. Olfactory marketing is increasingly used by several multinational companies to spread specified fragrances throughout their own environments. According to the present invention, such olfactory marketing is used for the first time as a means to promote the attention of the viewer at the terminal displaying an advertisement. In addition, the hosting facility can be a buyer of fragrances to be purchased from retailers, licensees, or suppliers of the remote terminals, because olfactory marketing can be used both to draw the interest of individuals toward the screens and as a means to increase the sales at the facility itself.

As a further illustration of certain technical details, only by means of an example, a computer system can be used that includes a central server (Main Server, MS) connected to the Internet 24 hours a day to deliver advertising messages, which central server has installed thereon a computer program for the management of such a function as well as all the administrative applications required by the different components of the platform.

In addition, the system includes one or more advertising display terminals (hereinafter simply referred to as ADTs) installed at commercial shops or other hosting locations. The ADT terminals can include a wireless networking peripheral device of either a Wi-Fi® type (internal mini PCI card) or a mobile HSDPA/UMTS/GPRS EDGE type (PCMCIA card), which provides for the connection of the terminal to the Internet. The ADTs can have installed an operating system which can run a minimal graphics management program, and the same ADT can include one or more video cameras integrated and/or either wired or wirelessly connected to the terminal along with people-counting software. The system can recognize and configure the networking peripheral device, and can open a browser at full screen that points to a Web page residing on the MS server to visualize both advertising messages and optional lure elements for the viewers such as news and information over time.

The computer system is designed for the interaction of several active and passive users of the system. Passive users are those attendees of facilities having installed ADT terminals who become targets of the advertisements delivered by the central system and who are analyzed by means of one or more video cameras in order to generate a statistical report of the effective hits.

Active users are those users who intentionally use the system, whether they are administrators, resellers, or advertisers. Administrators can monitor and manage the status of the application in real time, can use a Web interface to verify that management processes carried out by resellers have been successful, and in particular can drive and monitor revenue flow via the Internet. Resellers can both manage their own personal profile and plan the delivery of advertisements to the terminals associated therewith according to a schedule, can add one or more advertising messages of a given time length to each schedule (the advertising messages being textual messages, graphical messages or video clips with or without an audio track), and can determine which ADT terminals should be assigned to that schedule. Furthermore, each advertiser can set a maximum cost per hit for the advertisement to be delivered. General monitoring functions allow administrators, resellers, and advertisers to know the status of the terminals and to visualize statistics of usage for the terminals. Advertisers can receive a statistical report on the visualization of their advertising messages and the status of their active advertising campaigns.

In a particular embodiment the system includes a video camera and/or a hit analysis device that can be oriented and/or adjusted remotely.

In a particular embodiment the method includes: creating, setting, and submitting an advertising campaign; defining the characteristics of the recipients and the rules for the delivery of an advertising message; receiving the data of the campaign created by the advertiser at one or more servers; applying the criteria for the presentations of the advertising message to the schedule of one or more remote terminals; connecting the server to the remote terminals; presenting the advertising message on the remote terminals according to the parameters defined by the advertiser; periodically acquiring images of attendees; analyzing the attendees and determining the number of effective hits with respect to persons who have visualized the advertising message during a given time frame by means of a video camera; transmitting the acquired data to the server; analyzing and processing the acquired data; and charging the cost of the advertising message based both on the cost per hit and on the characteristics of the recipients as defined by the advertiser.

In a particular embodiment, the method can include combinations of the advertising message with a sound that can be heard preferentially by individuals belonging to a specified age group.

In a particular embodiment, the method can include the use of fragrances generated by the remote terminal to draw the attention toward the screen from which the advertising message is being delivered.

In a particular embodiment, the method can include a determination by the advertiser of the cost of an advertising message based on the age characteristics detected from the effective hits.

In a particular embodiment, the method can include a determination by the advertiser of the cost of an advertising message based on the gender detected from the effective hits.

In a particular embodiment, the method can include a determination of the cost of an advertising message based on the overall number of hits during a given time frame.

In a particular embodiment, the method can include an accounting of an error percentage in the number of detected hits, which error percentage is manageable by the administrator of the system and can be subtracted from the number of effective hits.

In a particular embodiment, the method can include presentation of an advertising message based on the effective hits during a specified time frame.

In a particular embodiment, the method can include analysis of attendees by counting the mobile phones or Bluetooth® devices within a specified operating range.

In a particular embodiment, the method can include analysis of attendees by a biometric recognition system to determine further useful characteristics of the individuals who visualize the advertising messages.

In a particular embodiment, the system includes the following computerized modules: a module for the creation of an advertising message; a module for reception of the advertising messages and determination of targets thereof; a module for selection of the advertising message, wherein the advertising message is selected according to the characteristics of the recipients; a module for delivery of the advertising message to a remote device; a module for presentation of the advertising message on the remote device; a module for transmission of sounds that can be heard preferentially by specified age groups; a module for the detection of the attendees; an analysis module for determination of the number of effective hits when the advertising message is visualized.

In a particular embodiment, the system can include a module that enables an advertiser to set a cost per hit in order to acquire a delivery priority over other competitors who are using a cost per hit which is lower than the cost per hit set by the advertiser.

In a particular embodiment, the module for the creation of an advertising message can implement a speech recognition method.

In a particular embodiment, the system can include a module for playing an audio-clip when a target of interest for the advertiser is in the proximity thereof.

In a particular embodiment, the system can include a module for playing a sound generated at specified frequencies.

Advantageously, the system can be characterized by: means for remotely creating and setting an advertising campaign; means for transmitting the advertising messages, said means being selected from a connection to the Internet, a telephone connection, or an SMS-capable network; means for receiving the advertising message; means for sending the advertising message to terminals through a modem and/or a data-transmission card connected to the network; means for receiving the advertising message at terminals provided at least with(i) a physical memory; (ii) a processor; (iii) an Internet-connection module; (iv) an operating system; (v) at least one video camera; (vi) a video card; (vii) people-counting software program having recognition capabilities; (viii) at least one application program to display the advertising messages; and (ix) means for transmitting the advertising messages to devices in the proximity of the terminals via Bluetooth®.

In a particular embodiment, the remotely manageable apparatus displaying the advertising messages and able to recognize the number of viewers can be characterized by a hardware arrangement comprising: a physical memory; a network card; at least one processor; at least one Internet-connection module; at least one supply connection; at least one operating system; a video card; a Wi-Fi® module; a Bluetooth® module; at least one video camera; people-counting computer program to determine the number and characteristics of the attendees; at least one application program to display the advertising messages; at least one screen; at least one database to save the data; at least one on/off switch that can be programmed remotely; at least one audio card to play sounds that can be heard preferentially by specified age groups and/or to play sounds adapted to mask background noise.

In a particular embodiment, the apparatus can include an audio card for playing sounds that can be heard preferentially by specified age groups.

In a particular embodiment, the apparatus can include an audio card for playing sounds that are adapted to mask the background noise.

In a particular embodiment, the apparatus can include one or more video cameras that can be connected wireless and/or oriented by means of a remote control and/or from a remote location.

In a particular embodiment, the apparatus can include an additional wireless mini-monitor to visualize how the individuals are remotely detected in order to minimize the amount of error.

In a particular embodiment, the apparatus can detect the existence of and send messages to devices that are in the proximity thereof and connected thereto via Bluetooth®.

### BRIEF DESCRIPTION OF THE DRAWINGS

FIG. 1 is a flow diagram of an exemplary method for advertising on remote terminals.

FIG. 2 is a block diagram of an exemplary system for advertising on remote terminals.

FIG. 3 is a block diagram of another exemplary system for advertising on remote terminals.

FIG. 4 is a flow diagram of another exemplary method for advertising on remote terminals.

FIG. 5 is a flow diagram of another exemplary method for advertising on remote terminals.

FIG. 6 is a block diagram of another exemplary system for advertising on remote terminals.

### DETAILED DESCRIPTION OF THE PREFERRED EMBODIMENTS

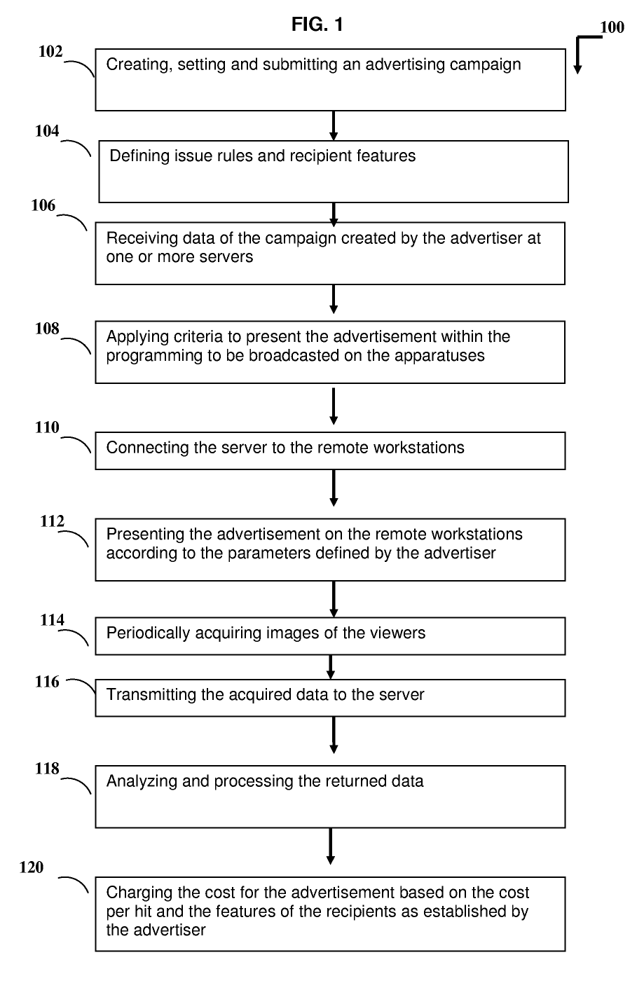

Referring to FIG. 1, a block diagram illustrates the method by which an advertiser connected to the Internet can create and set an advertising campaign 102 by defining the criteria for the delivery thereof and the characteristics of the recipients 104, such settings and preferences being received at a server 106. The server contains application programs that can apply the criteria for the delivery of the advertising campaign to the schedule of terminals 108. The advertising message is then visualized on a terminal provided with a screen and connected to the Internet network 110. The advertising message is presented according to defined parameters 112. Images of the attendees are periodically acquired 114. The resulting data are sent to the server 116. The images of the attendees are analyzed, and the number of individuals visualizing the advertising message is counted 118. The cost and the charge for the advertiser are determined based on those data 120.

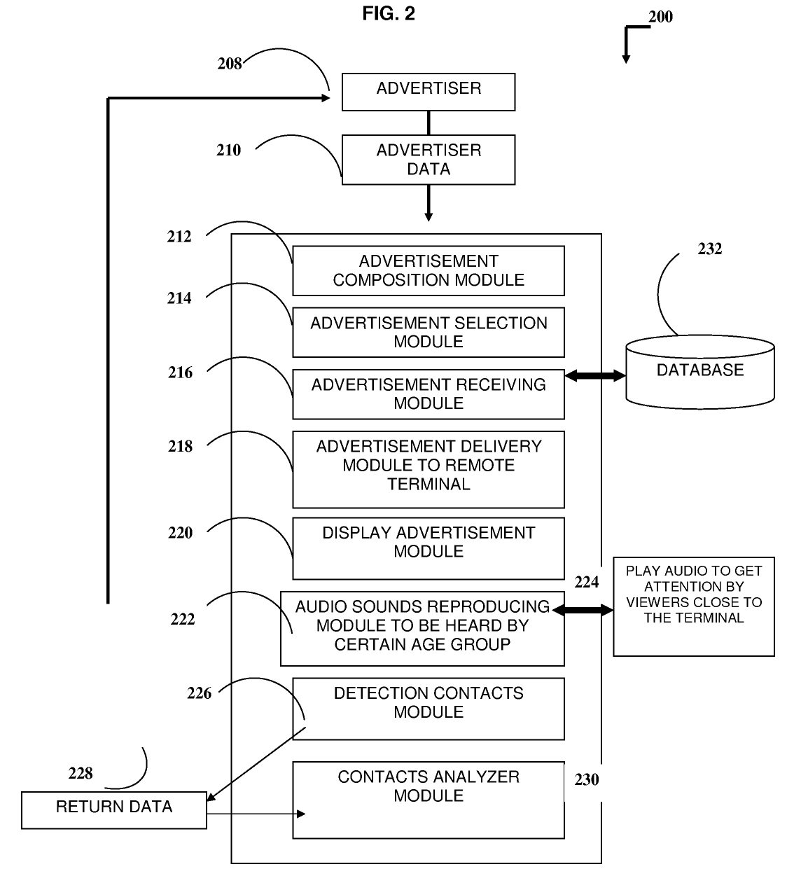

Referring to FIG. 2, a block diagram illustrates the system by which an advertiser 208 can create and deliver an advertising message 210. The system includes a message generation module 212, a message selection module 214, a message reception module 216 connected to a database 332, a message delivery module for transmitting the advertising message to a remote terminal 218, a message presentation module 220, a playback module for playing sounds at certain frequencies that can be heard preferentially by specified age groups 222 to draw the attention toward the terminal 224, and a people counting module 226. The detected data are sent to the server 230, and an attendee-analysis module 230 determines the number of attendees.

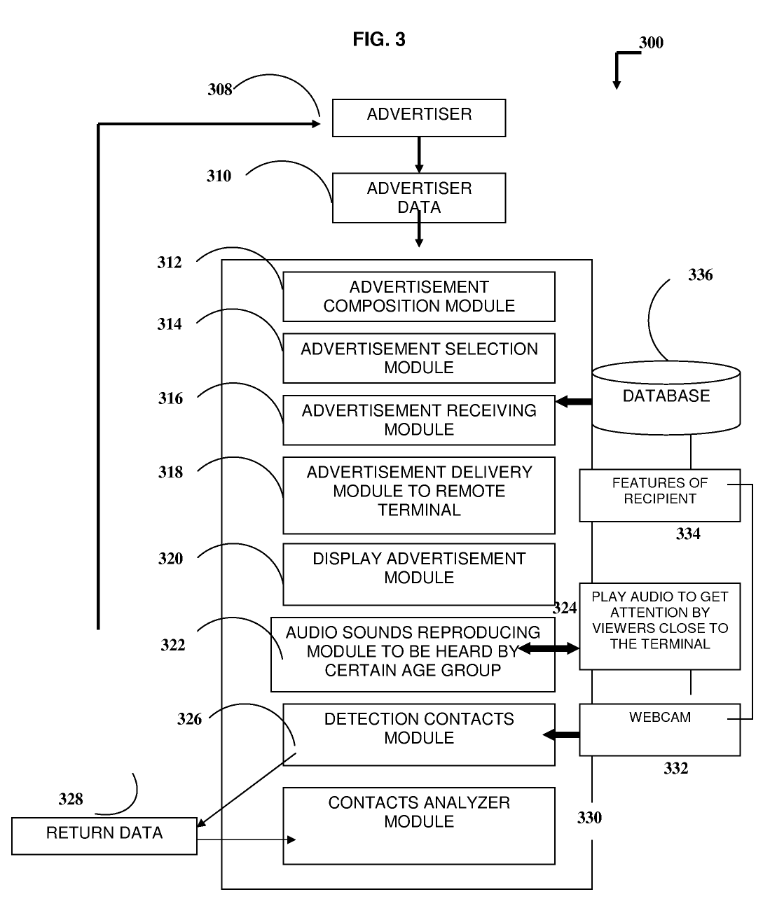

Referring to FIG. 3, a block diagram adds an alternative feature to the system, which alternative feature includes a people counting module provided with a video camera 332, which makes the advertising messages viewable according to the characteristics of the recipient 334, and which is interconnected to a database 336.

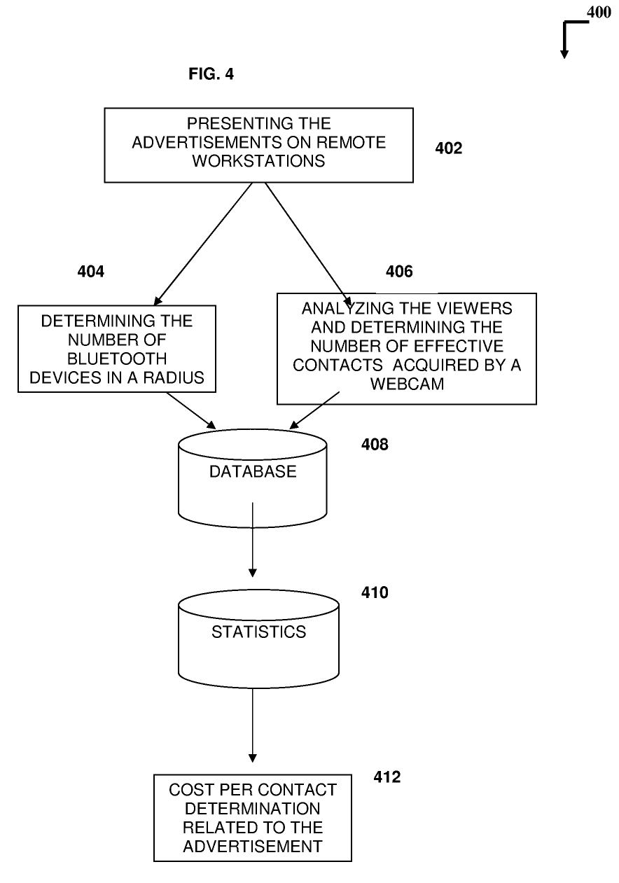

Referring to FIG. 4, a block diagram illustrates the method by which the number of hits can be determined with the use of two detection systems. The advertising messages are presented on remote terminals 402. Hits are detected by a combination of a video camera, a recognition program 406, and a verification of the existence of Bluetooth® devices within a surrounding area 404. The acquired information is transmitted to a database 408 and processed into statistical reports 410 used to determine the cost per hit for the advertisers 412.

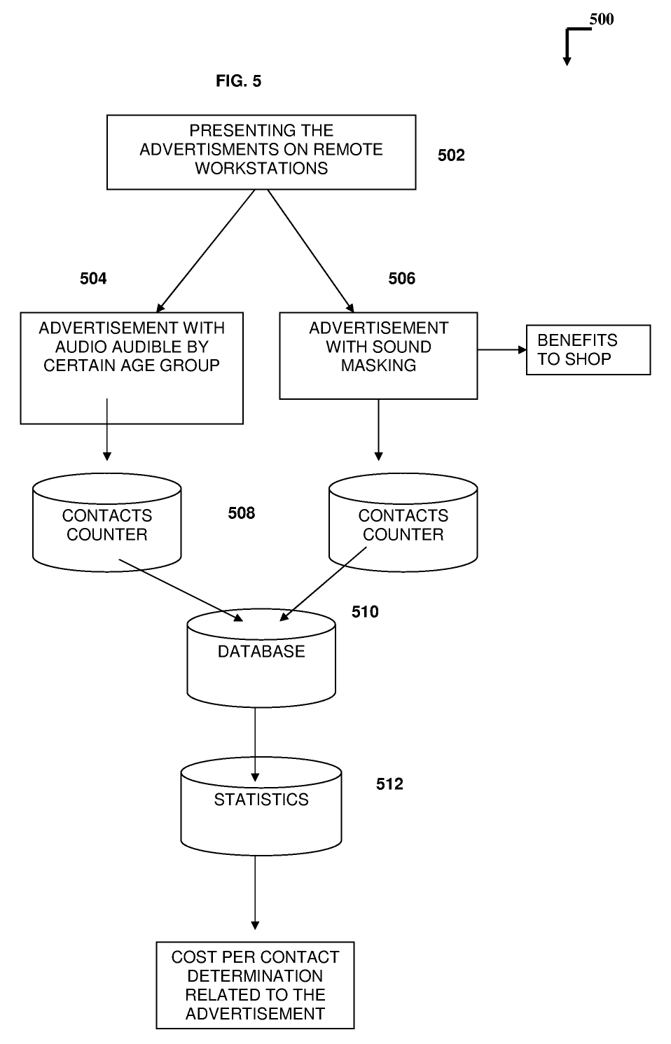

Referring to FIG. 5, a block diagram illustrates certain features. The advertising messages are presented on remote terminals 502. The advertising messages can be associated with an audio track that can be heard preferentially by specified age groups 504. Another audio track can also be generated to provide a sound-masking effect 506 which benefits the hosting facility. The attendees are counted 508 and data gathered into a database.

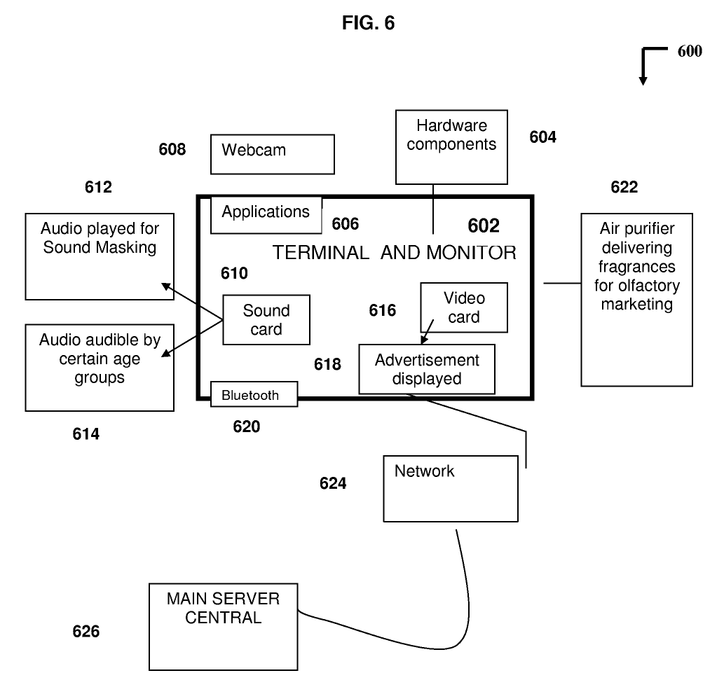

FIG. 6 is block diagram of the apparatus and the hardware portion thereof. The terminal provided with a screen 602 includes basic hardware components 604; application programs 606; at least one video camera 608; an audio card 610 for playing sounds which can apply a “sound masking” effect 612 and for playing sounds which can be heard preferentially by specified age groups 614; a video card 616=) for displaying the advertising messages 618. The apparatus includes a Bluetooth® module 620. The terminal is connected to an air purifier 622 that can deliver fragrances for the implementation of an olfactory marketing strategy. The apparatus is connected to a central server 626 through the network 624.

Although the systems and methods have been described with reference to many specific embodiments thereof, it is to be understood that the systems and methods are not limited to these embodiments, which are merely illustrative of some technical improvements of embodiments of systems to generate and set up an advertising campaign based on the insertion of advertising messages within an exchange of messages and corresponding methods to operate the systems. The disclosed systems and methods can be used with various devices and allows advertisers to exploit new paths to implement targeted advertising campaigns. Numerous industrial applications can be realized on a large scale or worldwide.

Those of skill further appreciate that the various illustrative logical blocks, configurations, modules, circuits, and algorithm steps described in connection with the embodiments disclosed herein may be implemented as electronic hardware, computer software, or combinations of both. To clearly illustrate this interchangeability of hardware and software, various illustrative components, blocks, configurations, modules, circuits, and features have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The methods or algorithms described in connection with the embodiments disclosed herein may be embodied directly in hardware, in a software module executed by a processor, or in a combination of the two. A software module may reside in random access memory (RAM), flash memory, read-only memory (ROM), programmable read-only memory (PROM), erasable programmable read-only memory (EPROM), electronically erasable programmable read-only memory (EEPROM), registers, hard disk, a removable disk, a compact disc (CD) ROM, or any other form of storage medium known or developed in the art. An exemplary storage medium is coupled to the processor such that the processor can read information from, and write information to, the storage medium. In the alternative, the storage medium may be integral to the processor. The processor and the storage medium may reside in an application specific integration circuit (ASIC). The ASIC may reside in a computing device, a user terminal, or a telephone. In the alternative, the processor and the storage medium may reside as discrete components in a computing device, user terminal, or telephone.

The previous description of the disclosed embodiments is provided to enable any person skilled in the art to make or use the disclosed embodiments. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope possible consistent with the principles and features as defined by the following claims.

In the foregoing Summary and Detailed Description, various features may be grouped together in several exemplary embodiments for the purpose of streamlining the disclosure. This method of disclosure is not to be interpreted as reflecting an intention that any claimed embodiment requires more features than are expressly recited in the corresponding claim. Rather, as the appended claims reflect, inventive subject matter may lie in less than all features of a single disclosed exemplary embodiment. Thus, the appended claims are hereby incorporated into the Detailed Description, with each claim standing on its own as a separate disclosed embodiment. However, the present disclosure shall also be construed as implicitly disclosing any embodiment having any suitable combination of disclosed or claimed features that appear in the present disclosure or the appended claims (i.e., combinations of features that are not incompatible or mutually exclusive), including those combinations of features that may not be explicitly disclosed herein. It should be further noted that the scope of the appended claims do not necessarily encompass the whole of the subject matter disclosed herein.

What is claimed is:

1) A method to pay for an advertisement, which advertisement is presented by remotely manageable workstations, according to the number of viewers, comprising:
creating, setting, and submitting an advertising campaign;
defining issue rules and recipient features;
receiving data of the campaign created by an advertiser at one or more servers;
applying criteria to present the advertisement within programming to be broadcasted on the remote workstations;
connecting the server to the remote workstations;
presenting the advertisement on the remote workstations according to the defined issue rules and recipient features;
periodically acquiring images of viewers of the advertisement;
analyzing the images of the viewers and determining a number of effective hits by persons who have visualized the advertisement within a time period through a video camera;
transmitting acquired data to the server;
analyzing and processing the transmitted data;
charging a cost for the advertisement based on the cost per hit and the features of the viewers as established by the advertiser.

2) The method of claim 1, wherein the advertisement is provided with a sound that is heard preferentially by viewers belonging to a certain age group.

3) The method of claim 1, wherein one or more fragrances are generated by the workstation, which fragrances are remotely selected by the advertiser to stimulate the interest towards the workstation broadcasting the advertising.

4) The method of claim 1, wherein hits are determined by using the data acquired by analyzing the images of the viewers and by counting Bluetooth® devices existing within a specific radius.

5) The method of claim 1, wherein the advertiser sets a cost per hit and, according to said cost per hit, thereby acquires an issue priority over other advertisers using a lower cost per hit.

6) The method of claim 1, wherein a specific advertisement is presented according to the features of the viewers or a hosting environment.

7) The method of claim 1, wherein determining the number of effective hits comprises determining an overall number of viewers within a time period and a number of unique viewers during presentation of the advertisement.

8) A system to detect the effective hits for advertising campaigns which can be presented by remote workstations connected through a computer network, comprising:
a module to create an advertisement;
a module to select the advertisement, wherein the advertising is selected according to recipient features;
a module to receive the advertisements and determine the recipients thereof;
a module to deliver the advertisement to the remote workstations;
a module to present the message on the remote device;
a module to play sounds that can be heard preferentially by certain age groups;
a module to detect viewers;
an analyzer module to determine the number of effective hits while the advertisement is being presented.

9) The system of claim 8, wherein the recipient features of the advertisements are determined based on information received from a video camera.

10) The system of claim 8 further comprising an audio player module.

11) A remotely manageable apparatus to present advertisements, which apparatus is structured and connected to determine a number of viewers of the advertisements, the apparatus comprising: a physical memory; a network card; at least one processor; at least one module to connect to the Internet network; at least one power connection; at least one operating system; a video card; a WI-FI module; a Bluetooth® module; at least one wired or wireless video camera that is remotely manageable; at least one person-counting computer program to count the viewers and determine the features thereof; at least one display module to present advertisements; at least one monitor; at least one database to save data; at least one on/off switch with remote programming functionality; at least one audio card to play sounds that are heard preferentially by certain age groups or to play sounds suitable to mask background noise.

12) The apparatus of claim 11 further comprising an air purifier/ionizer structured and connected to deliver fragrances to stimulate the interest of the viewer towards the monitor broadcasting the advertisement for olfactory marketing actions.
